(19) United States
(12) Patent Application Publication
(10) Pub. No.: US 2010/0274231 A1
(43) Pub. Date: Oct. 28, 2010

(54) RENAL FLUSHING CATHETER

(75) Inventors: Boun Pravong, Corona, CA (US); Charles C. Hart, Summerville, NC (US); Ghassan Sakakine, Rancho Santa Margarita, CA (US); Eric Nguyen, Corona, CA (US)

Correspondence Address: APPLIED MEDICAL RESOURCES CORPORATION, 22872 Avenida Empresa, Rancho Santa Margarita, CA, 92688, US

(73) Assignee: Applied Medical Resources Corporation, Rancho Santa Margarita, CA (US)

(21) Appl. No.: 12/765,263

(22) Filed: Apr. 22, 2010

Related U.S. Application Data
(60) Provisional application No. 61/172,586, filed on Apr. 24, 2009.

(51) Int. Cl. A61B 17/22

(52) U.S. Cl. 606/2.5; 604/96.01

(57) ABSTRACT

Devices, methods, and systems provide a catheter-based device comprising an expandable portion that is placed distally of the obstructive or occluding mass, which substantially prevents fragments generated by ablating the occluding mass with a lithotripter, for example, a laser lithotripter, from traveling upstream, thereby improving the safety and effectiveness of lithotripsy Some embodiments of the device further comprise a through lumen through which fluid is introduced into a renal collecting system, thereby maintains positive pressure that biases the fragments generated by lithotripsy from entering the collecting system.

### CROSS-REFERENCE TO RELATED APPLICATIONS

This application claims the benefit of U.S. Provisional Application No. 61/172,586, filed Apr. 24, 2009, the entire disclosure of which is hereby incorporated by reference as if set forth in full herein

### BACKGROUND

This disclosure is related to medical devices, and more particularly, to a renal flushing catheter useful in lithotripsy of ureteral calculi.

Devices that assist in the removal or retrieval of ureteral calculi or stones generally comprise basket-like structures that are sized and configured to capture such a mass so that it can be mobilized or held in position.

However, there remains a need for a device that facilitates the use of a directed energy source, such as a laser, to fragment a luminal mass, such as a urinary stone, by specifically blocking fragments from moving upstream in the ureter, that is, in the direction of the directed energy. In addition, there is a need for a device that biases a fluid flow through the ureter, thereby propelling fragments in a retrograde direction.

### SUMMARY

Devices, methods, and systems provide a catheter-based device comprising an expandable portion that is placed distally of the obstructive or occluding mass, which substantially prevents fragments generated by ablating the occluding mass with a lithotripter, for example, a laser lithotripter, from traveling upstream, thereby improving the safety and effectiveness of lithotripsy. Some embodiments of the device further comprise a through lumen through which fluid is introduced into a renal collecting system, thereby maintains positive pressure that biases the fragments generated by lithotripsy from entering the collecting system.

In one embodiment, a catheter based occlusive device for use within a body lumen to maintain a gradient fluid pressure and form a luminal blockage is provided. The device comprises an elongate luminal catheter body having a lumen extending from a proximate end to a distal end of the catheter body and a plurality of expandable portions near the distal end of the catheter body. In the low-profile state, each of the expandable portions has a crossing profile sufficient to permit placement of the expandable portion distal of a ureteral mass, and in the high-profile state, each of the expandable portions substantially block fragments generated by lithotripsy of the ureteral mass from traveling distally thereof.

In another embodiment, a catheter based occlusive device for use within a body lumen to maintain a gradient fluid pressure and form a luminal blockage is provided. The catheter comprises an elongate luminal catheter body having a lumen extending from a proximate end to a distal end of the catheter body and a plurality of expandable portions attached to the distal end of the catheter body. The plurality of expandable portions has a low-profile state and a high-profile state and the lumen of the catheter body provides a fluid conduit to inject irrigation fluid out the distal end of the catheter body and distally from the plurality of expandable portions. The plurality of expandable portions in the high-profile state obstructs fragments of a ureteral mass from traveling distally thereof and does not obstruct the fluid conduit.

Many of the attendant features of the present invention will be more readily appreciated as the same becomes better understood by reference to the foregoing and following description and considered in connection with the accompanying drawings in which like reference symbols designate like parts throughout.

### BRIEF DESCRIPTION OF THE DRAWINGS

FIG. 1 illustrates a common urinary tract, urinary bladder, ureters, and kidneys.

FIG. 2A illustrates a common urinary tract obstruction in a ureter and an embodiment of a renal flushing catheter in a low-profile state in place within the ureter. FIG. 2B illustrates the renal flushing catheter illustrated in FIG. 2A comprising a plurality of inflatable balloons in an expanded state. FIG. 2C is a perspective view of an embodiment of a distal end of the catheter illustrated in FIGS. 2A and 2B in the expanded state. FIG. 2D is a detailed view of the upper portion of FIG. 2C illustrating directing energy upon a luminal obstruction.

FIG. 3 illustrates an embodiment of a collection vessel.

FIG. 4A is a perspective view of an expandable portion of another embodiment of a renal flushing catheter comprising a radially expandable mesh expandable portion in a low-profile state. FIG. 4B is a perspective view of the expandable portion illustrated in FIG. 4A in a high-profile state FIG. 4C illustrates the renal flushing catheter illustrated in FIGS. 4A and 4B with the expandable portion in the expanded state placed distally of a occluding mass.

FIG. 5A is perspective view of an expandable portion of another embodiment of a renal flushing catheter comprising a plurality of cone-shaped members in a low-profile state. FIG. 5B is perspective view of the expandable portion illustrated in FIG. 5A in a high-profile state. FIG. 5C illustrates the renal flushing catheter illustrated in FIGS. 5A and 5B with the expandable portion in the expanded state placed distally of a occluding mass.

FIG. 6 illustrates another embodiment of a renal flushing catheter comprising an expandable portion that is cone-shaped in an expanded state.

FIG. 7A is a perspective view of an expandable portion of another embodiment of a renal flushing catheter comprising a convoluted mesh expandable portion in a low-profile state. FIG. 7B is a perspective view of the expandable portion illustrated in FIG. 7A in a high-profile state. FIG. 7C illustrates another embodiment of a renal flushing catheter comprising a convoluted mesh structure. FIG. 7D is a detailed view of the upper portion of FIG. 7C illustrating directing energy upon a luminal obstruction.

FIG. 8 illustrates another embodiment of a renal flushing catheter in which an expandable portion comprises a linearly compressible woven fabric.

### DETAILED DESCRIPTION

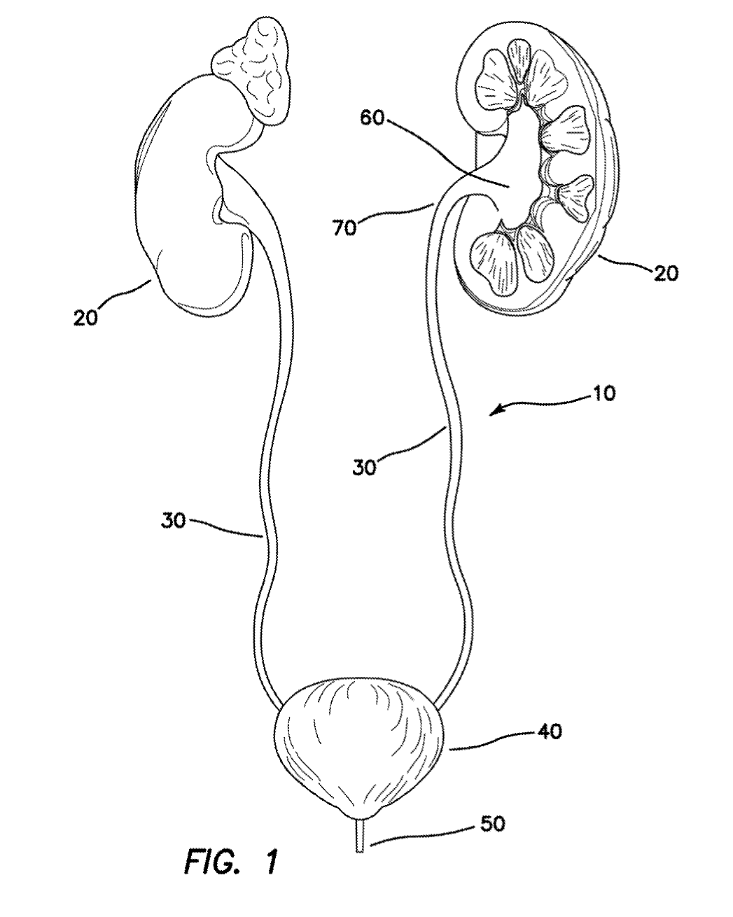

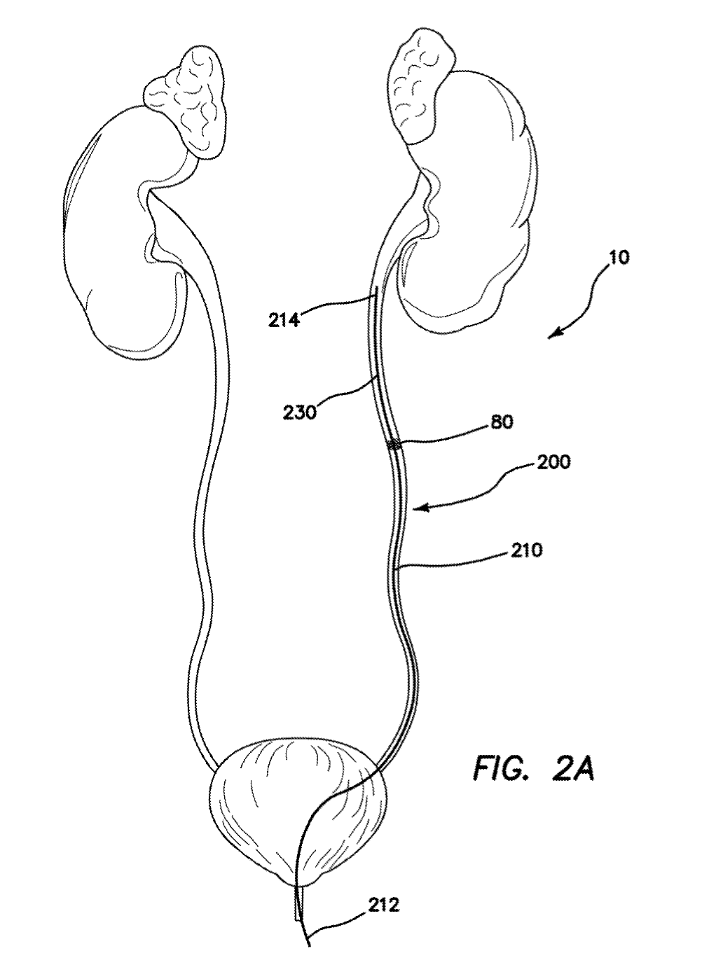

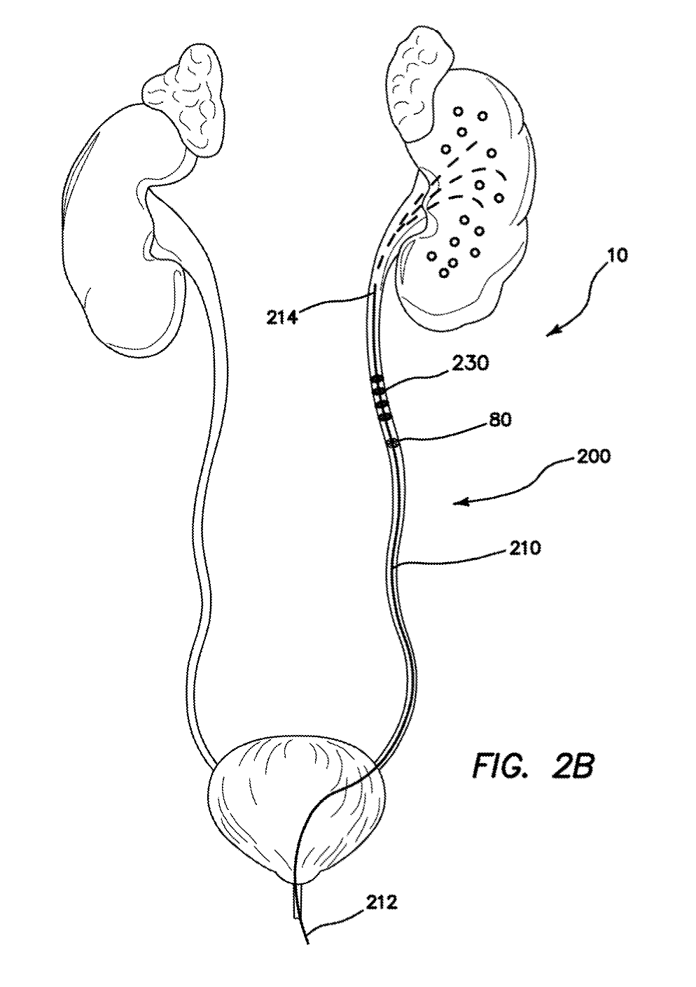

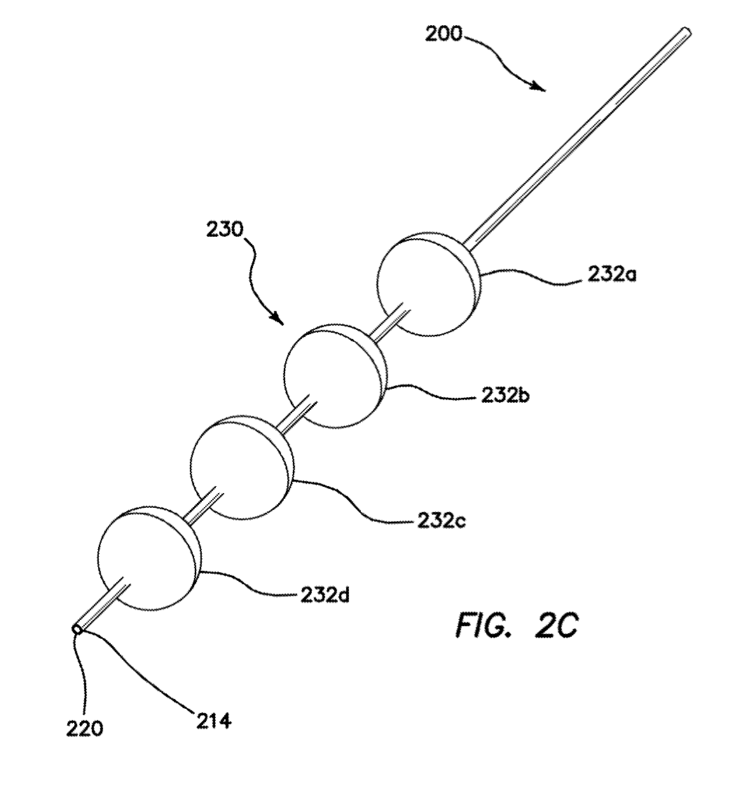

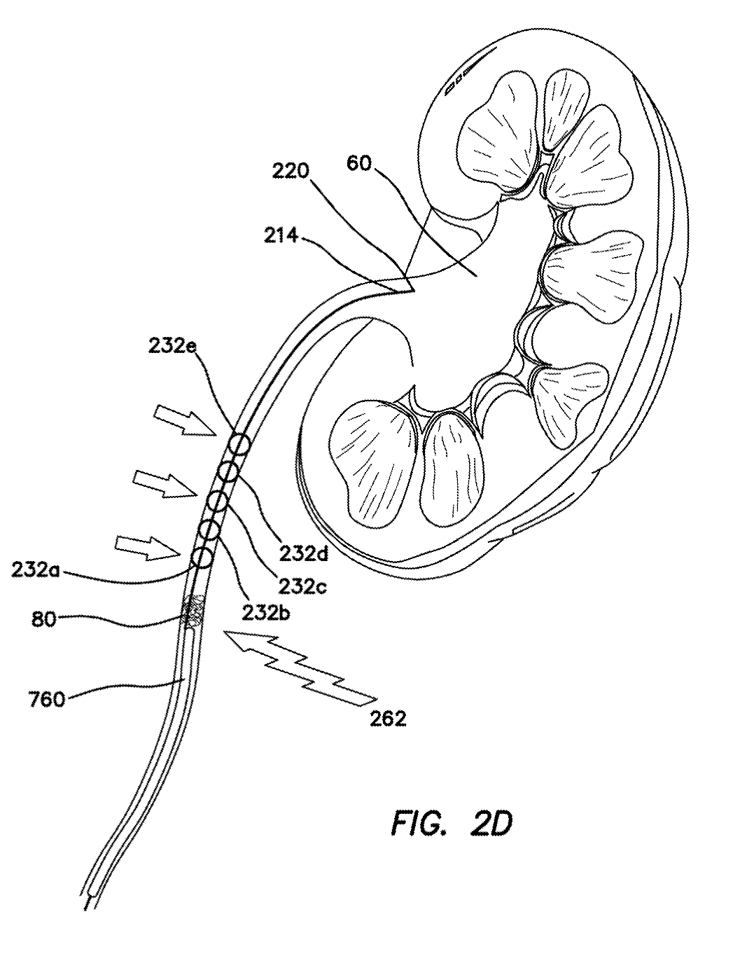

FIG. 1 illustrates a urinary tract 10 comprising a urinary bladder 40, a pair of ureters 30, and a pair of kidneys 20. As shown in FIG. 2A, occasionally, a mass 80 forms within the urinary tract 10 and becomes lodged within the lumen of the ureter 30, thereby obstructing or occluding the ureter 30. The mass 80 may be very solid and tractive within the lumen. Because the mass 80 will continue to grow and eventually completely block the ureter 80, it is necessary to remove or dissolve such a mass. The mass 80 is fragmented or ablated using a lithotripter, which comprises an energy source that emits directed energy suitable for fragmenting the mass 80. The resulting fragments are then removed from the lumen of the ureter 30. Suitable energy sources typically generate a localized shock wave that mechanically fragments the mass, for example, at least one of ultrasonic sound waves, electrically-generated shock waves, or laser energy. Those skilled in the art will appreciate that delivering strong, focused energy to the mass 80 within a ureteral lumen 30 can move the mass 80 and/or fragments thereof upstream, that is, back toward the renal collecting system 60, which is a very undesirable outcome. Fragments in the collecting system 60 are not typically flushed therefrom and seed additional calculi.

In some cases, a physician deploys a holding device that can capture or contain the mass 80 and hold it securely while it is fragmented by the lithotripter. Such procedures are sometimes problematic, however, especially using laser lithotripsy, because the holding device may be damaged and/or destroyed in the lithotripsy process. For example, where the holding device comprises a metallic wire “basket”, portions of the basket surviving the lithotripsy may present a serious problem for removal of the device. An inflatable balloon placed behind the mass may provide a better option; however, the lithotripsy energy can rupture the balloon, after which fragments thereof may then move upstream.

FIG. 2A illustrates an embodiment of a renal flushing catheter 200 in a low-profile state positioned in a urinary tract 10. The renal flushing catheter 200 comprises an elongate body 210 comprising a proximal end 212 and a distal end 214. The distal end 214 is sized and configured to easily pass alongside the ureteral mass 80 in the undeployed, low-profile state when the catheter 200 is positioned, as illustrated in FIG. 2A. The distal end 214 comprises an expandable occluding portion 230. The proximal end 212 comprises an activation member, the use of which is discussed below.

Some embodiments of the elongate body 210 comprise two coaxial, tubular members that are relatively slidable Relatively sliding the coaxial, tubular members applies or releases tension on the expandable member 230, thereby converting the expandable portion 230 between the low-profile state illustrated in FIG. 2A and a high-profile state illustrated in FIG. 2B. In other embodiments, the elongate body 210 comprises an inflation lumen through which applying or releasing pressure converts the expandable member 230 between the low-profile state and the high-profile state. Some embodiments of the elongate body 210 comprise one or more additional lumens, in which, for example, at least a portion of a visualization system is disposed, which is used during placement of the catheter 200 and/or fragmentation of the mass 80. In some embodiments, at least a portion of a lithotripsy or energy delivery system is disposed in the one or more additional lumens.

The expandable portion 230 is characterized by a “crossing profile”, which is defined as a largest diameter of the expandable portion 230 in the low-profile state. A smaller crossing profile facilitates positioning the distal end 214 of the catheter around the mass 80, particularly, in cases in which the mass 80 is large, and/or the lumen of the ureter 30 has a small diameter. In some embodiments, the crossing profile of the expandable portion 230 is not greater than about 2 mm, 1.6 mm, 1.35 mm, 1 mm, or 0.65 mm (about 0.025″) In some embodiments, the crossing profile of the expandable portion 230 is not greater than about 4 French, 3 French, or 2 French. In some embodiments, the expandable portion 230 comprises one or more expandable features that have diameters greater than the crossing profile when the expandable portion 230 is in the high-profile state. In some embodiments, a diameter of an expandable feature in the high-profile state is at least about 0.9 mm (about 0.035″), 5 mm, 6 mm, or 7 mm. In some embodiments, the diameter of an expandable feature in the high-profile state is at least about 6 French, 8 French, 15 French, or 17 French. Embodiments of the expandable portion 230 resist failure during ablation of the mass 80, for example, using a laser. For example, embodiments of the expandable portion 230 are configured and constructed such that laser energy incident on the expandable portion 230 in the high-profile state affects only relatively small portions thereof, thereby maintaining an occluding “backstop” upstream of any portion of the expandable portion 230 that are ablated by the directed energy.

FIG. 2C is a perspective view of the distal end 110 of the catheter 200 illustrated in FIG. 2B in which the expandable portion 230 is in the high-profile state In the illustrated embodiment, the expandable feature of the expandable portion 230 comprises a plurality of inflatable balloons 232a-232d. In the low-profile state, for example, as shown in FIG. 2A, the balloons 232a-232d present a low crossing profile. A through lumen 220 opens at the extreme distal end 214 of the catheter body 210 in the illustrated embodiment. The balloons 232a-232d are distensible. Some embodiments of the balloons comprise at least one of an elastomeric polymer, polyurethane, polyisoprene, styrene-butadiene, silicone, ethylene-propylene rubber, and the like. Some embodiments of the balloons are fenestrated.

In use, the catheter 200 is positioned in the urinary tract 10 with the expandable portion 230 in the low profile state, as illustrated in FIG. 2A. In some embodiments, the progress of the distal end 214 relative to the ureteral mass 80 is monitored using a visualization system. After the expandable portion 230 has been advanced past the mass 80, the expandable portion 230 is converted into the high profile state using the activation member.

FIG. 2D is a detailed view of the upper portion of the ureter and kidney illustrated in FIG. 2B with the catheter 200 deployed therein. In the illustrated embodiment, the activation member pressurizes the inflation lumen, thereby inflating the individual balloons 232a-232e upstream of the ureteral mass 80. In some embodiments, the inflation is visually monitored. An irrigating fluid is then delivered under low pressure through the through-lumen 220 upstream of the balloons 232a-232e, thereby partially filling the collecting system 60 of the kidney. The balloons 232a-232e seal the ureter, thereby retaining the fluid in the collecting system 60. In one embodiment, the balloons in a high-profile and/or low-profile state do not obstruct the through-lumen 220 and/or a fluid conduit through which the irrigating fluid is introduced. In another embodiment, the balloons obstruct or seal the through-lumen and/or fluid conduit when the balloons are in a high-profile state. A lithotripter 260, for example, a laser lithotripter, is then be “fired” or activated, which delivers a pulse 262 of directed energy that fragments or ablates the ureteral mass 80 using the most proximal, balloon 232a as a “backstop”. An example of a suitable laser lithotripter includes a holmium laser lithotripter. Any energy not absorbed by the ureteral mass 80, for example, from a misfire, is likely to pop the balloon 232a, which deflates. The next, redundant balloon 232b now serves as the “backstop” for the next firing of the laser 260. The sequence may be repeated until the ureteral mass 80 is fragmented and all of the balloons 232 are deflated and/or destroyed, thereby releasing the irrigation fluid from the renal collecting system 60. In one embodiment, one or more of the expandable portions, e.g., the proximate balloon 232a, is destructible or susceptible to destruction by, for example, a laser lithotripter, and one or more of the other expandable portions, e.g., distal balloon 232e, is not destructible or more resistant to destruction. With the balloons 232 in a low profile state, the fluid carries the ablated fragments proximally or downstream.

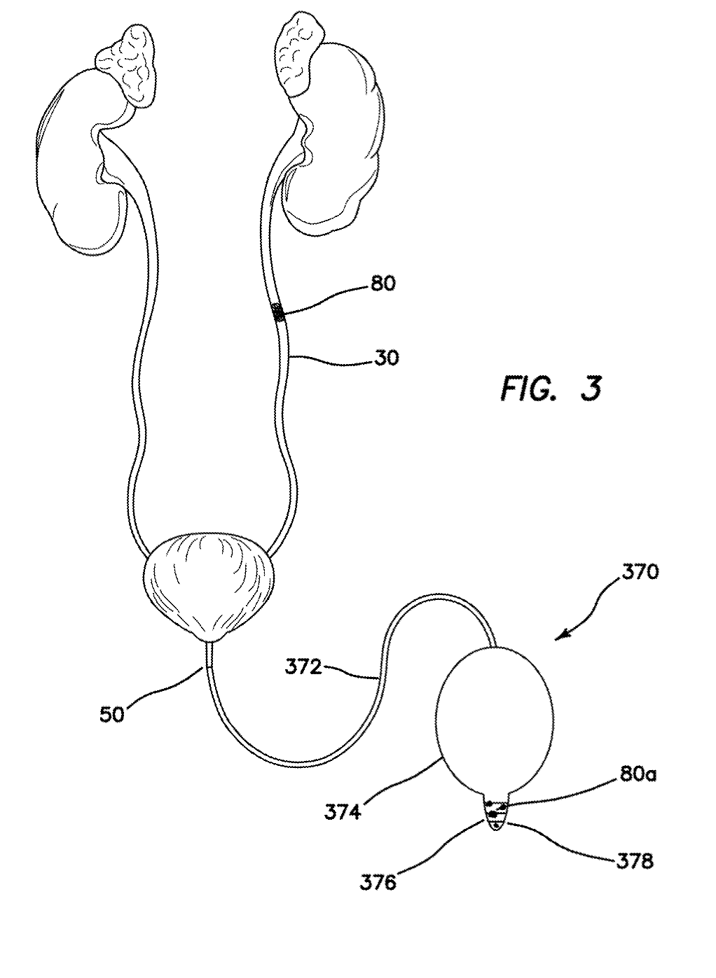

FIG. 3 illustrates an embodiment of a collection vessel 370 fluidly connected to the proximal end 50 of the urinary tract through a tube 372. In the illustrated embodiment, the collection vessel 370 comprises a transparent tapered collection bag 374 comprising a bottom portion 376 dimensioned and configured as a collection reservoir. In the illustrated embodiment, the bottom portion 376 comprises calibrated indicia 378, thereby permitting a user to assess and compare the fragment mass 80a with the estimated pre-operative mass of the occluding mass 80 within the ureter 30.

In some embodiments, a ureteral stent is then placed to maintain the patency of the ureter 30. In one embodiment, the ureteral stent is removable and placed around the catheter body surrounding all or a portion of the plurality of expandable balloons or portions of the catheter. In some embodiments, the catheter 100 serves as a guidewire over which the stent is inserted. The catheter 100 is then withdrawn.

In other embodiments, a guidewire is advanced through the through lumen 220 of the catheter and catheter 100 is withdrawn. The stent is then advanced over the guidewire and placed. The guidewire is then withdrawn.

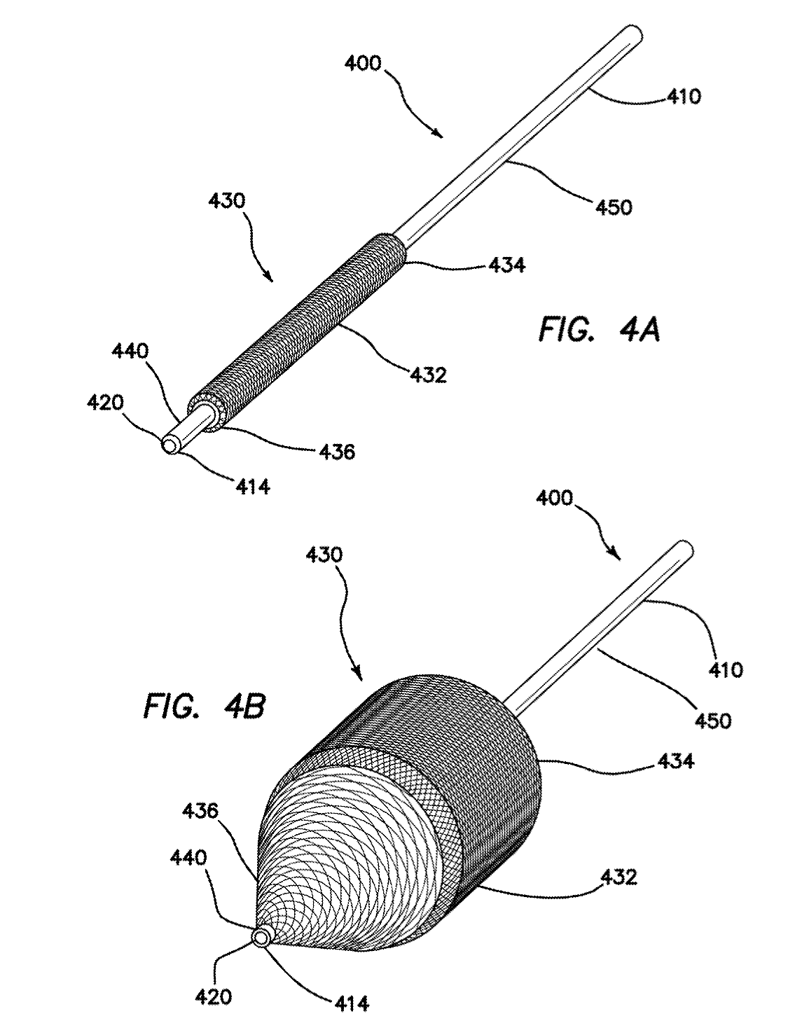

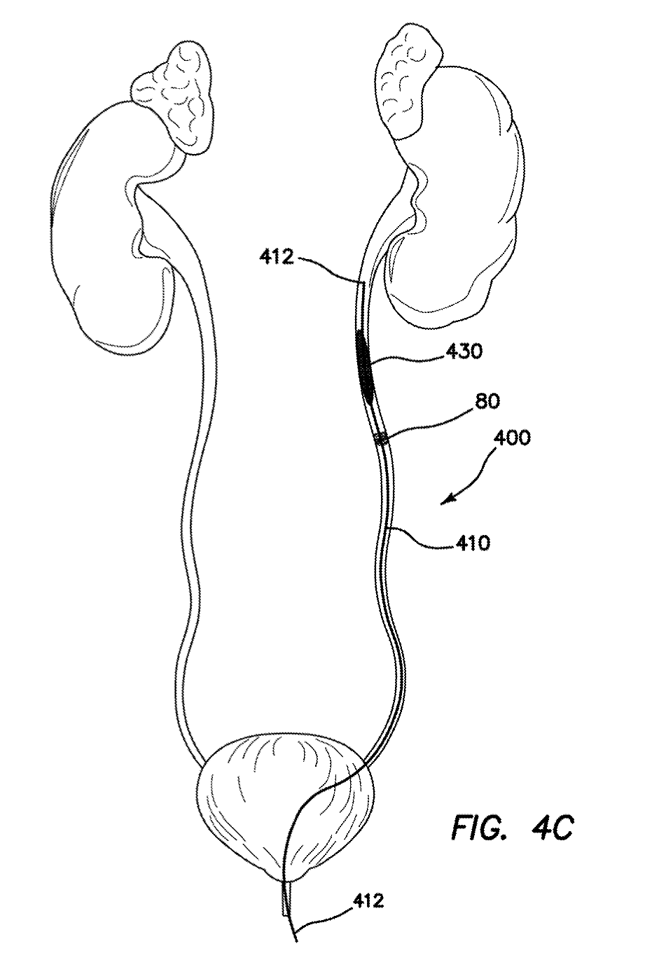

FIG. 4A is a perspective view of another embodiment of an expandable portion 430 of a renal flushing catheter 400 in a low-profile state, and FIG. 4B is a perspective view of the expandable portion 430 in a high-profile state. The renal catheter 400 is generally similar to the embodiment discussed above and illustrated in FIGS. 2A-2D. In the illustrated embodiment, the catheter 400 comprises a catheter body 410 comprising a proximal end 412 and a distal end 414. A through-lumen 420 extends through the catheter body 410, terminating at the distal end 414. In the illustrated embodiment, the catheter body 410 comprises an inner tube 440 and an outer tube 450, which are coaxial and relatively slidable longitudinally. In some embodiments, the inner tube 440 and outer tube 450 are also relatively rotatable. The relative positions, longitudinal and/or rotational, between the inner tube 440 and outer tube 450 are controlled by an activation member disposed at the proximal end 412 of the catheter.

As discussed above, the expandable portion 430 is disposed proximate to the distal end 414 of the catheter. In the illustrated embodiment, the expandable portion 430 comprises a cylindrical, braided fabric element 432, which comprises a proximal end 434 and a distal end 436. The distal end 436 of the fabric element is coupled with the inner tube 440 of the catheter body, and the proximal end 434 is coupled to the outer tube 450. Because the inner tube 440 and outer tube 450 are relatively slidable, adjusting their relative positions compresses or tensions the fabric element 432. Those skilled in the art will understand that in other embodiments, the inner tube 440 is coupled to the proximal end 434, and the outer tube 450 is coupled to the distal end 436.

In some embodiments, a diameter of the braided fabric element 432 varies predictably with its length. In the embodiment illustrated in FIGS. 4A and 4B, the expandable portion 430 is maintained in a desired crossing profile by applying tension upon the braided element 430 through the inner tube 440 and the outer tube 450. In some embodiments, the expandable portion 430 is expanded by adjusting and/or releasing the applied tension to provide a generally cylindrical portion 430 in the high-profile state with a larger diameter and a shorter length compared with the dimensions in the low profile state. In some embodiments, the expandable portion 430 is further expanded or deployed by at least one of linearly compressing, twisting, and/or otherwise deforming the braided element 432, for example, by relatively adjusting the positions of the inner tube 440 and the outer tube 450. In some embodiments, the degree of expansion of the braided element 432 is visually monitored. FIG. 4C illustrates a urinary tract in which the renal flushing catheter 400 is placed with the expandable portion 430 deployed upstream of a ureteral mass 80.

Embodiments of the braided mesh fabric comprise at least one of a polymer, polymer, polyester, polyamide, polytetrafluoroethylene, and polyurethane. The braided mesh fabric comprises a plurality of bias-woven fibers, and as such, is damage tolerant because damage to a few fibers will not cause the structure to fail. In some embodiments, the braided element 430 in the high-profile state permits a degree of fluid flow therethrough, thereby reducing the chance of over pressurizing the collection system 60.

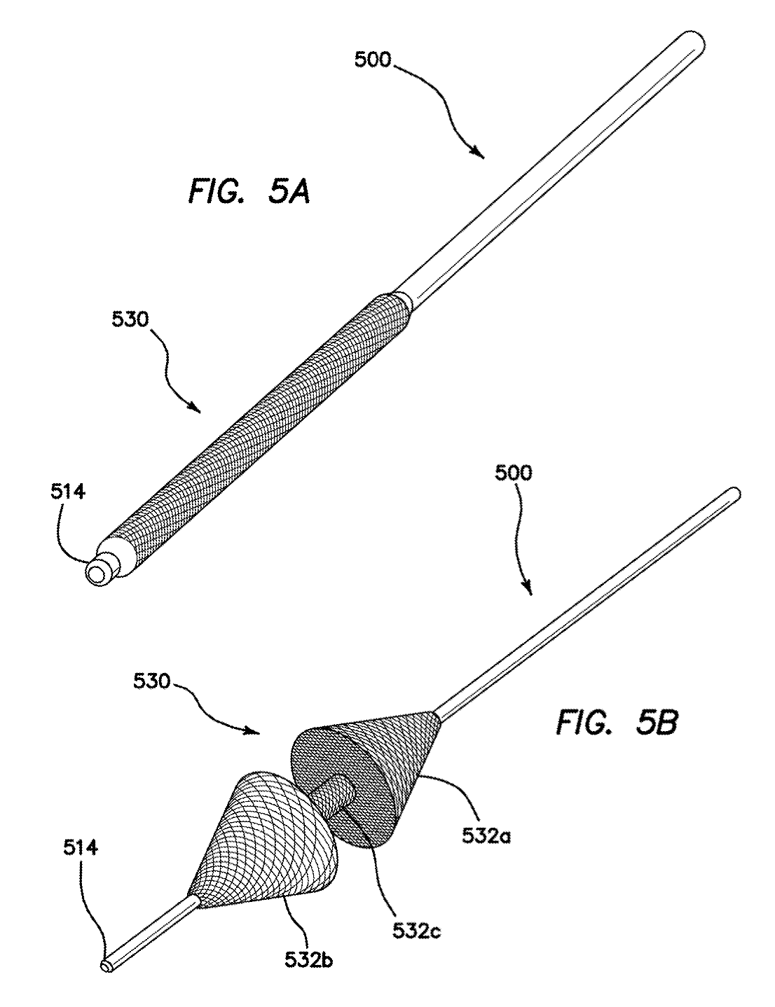

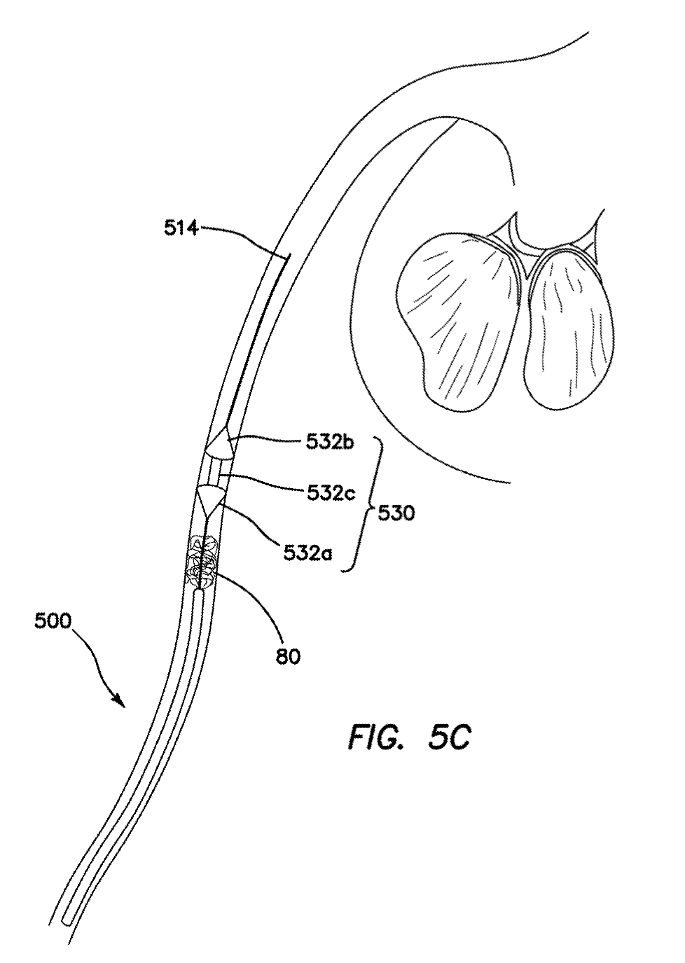

FIG. 5A is a perspective view of a distal end 514 of another embodiment of a renal flushing catheter 500 in a low-profile state. FIG. 5B is a perspective view of the distal end 514 in a high-profile state. The catheter 500 illustrated in FIGS. 5A and 5B is generally similar to the embodiments described above, and illustrated in FIGS. 4A-4C. An expandable portion 530 comprises a cylindrical braided mesh element 532 in the low-profile state. In the high profile state, the expandable portion 530 comprises a proximal cone 532a, a distal cone 532b, and a cylindrical portion 532c disposed therebetween. In the illustrated embodiment, the proximal cone 532a points proximally and the distal cone 532b points distally. In the illustrated embodiment, the proximal cone 532a and the distal cone 532b are generally symmetrical, with similar diameters and lengths. A diameter of the cylindrical portion 532c is smaller than the largest diameters of the proximal cone 532a and the distal cone 532b. FIG. 5C is a view of an upper portion of a urinary tract with the catheter 500 placed therein with the expandable portion 530 deployed distally of a ureteral mass 80. In one embodiment, each portion can be individually or group manipulated, deformed and/or destroyed to reversibly move from a low-profile to a high-profile state. In various other embodiments, one or more portions have a different material characteristic, structure and/or placement relative to the other portions or catheter to assist in the reversible deployment or expansion of the portions to obstruct or not obstruct fluid flow and/or the ureteral mass or portion thereof.

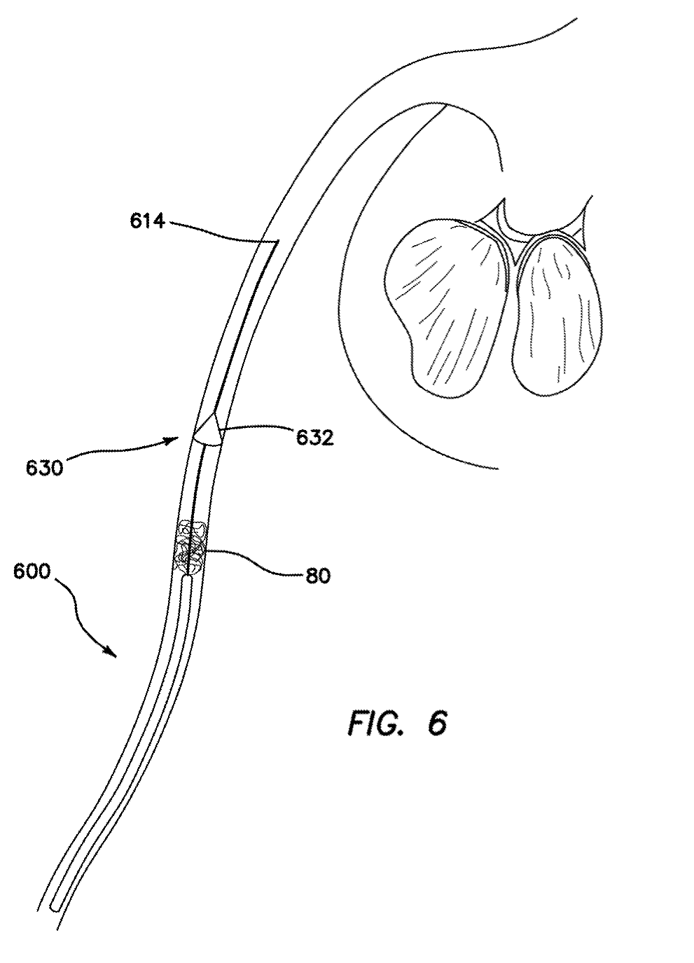

FIG. 6 is a view of an upper portion of a urinary tract with another embodiment of a catheter 600 placed therein with an expandable portion 630 deployed distally of a ureteral mass 80. The catheter 600 is generally similar to the embodiments described above and illustrated in FIGS. 5A and 5B except that in the high-profile state, the expandable portion 630 comprises a braided mesh 632 in the shape of a cone pointed distally.

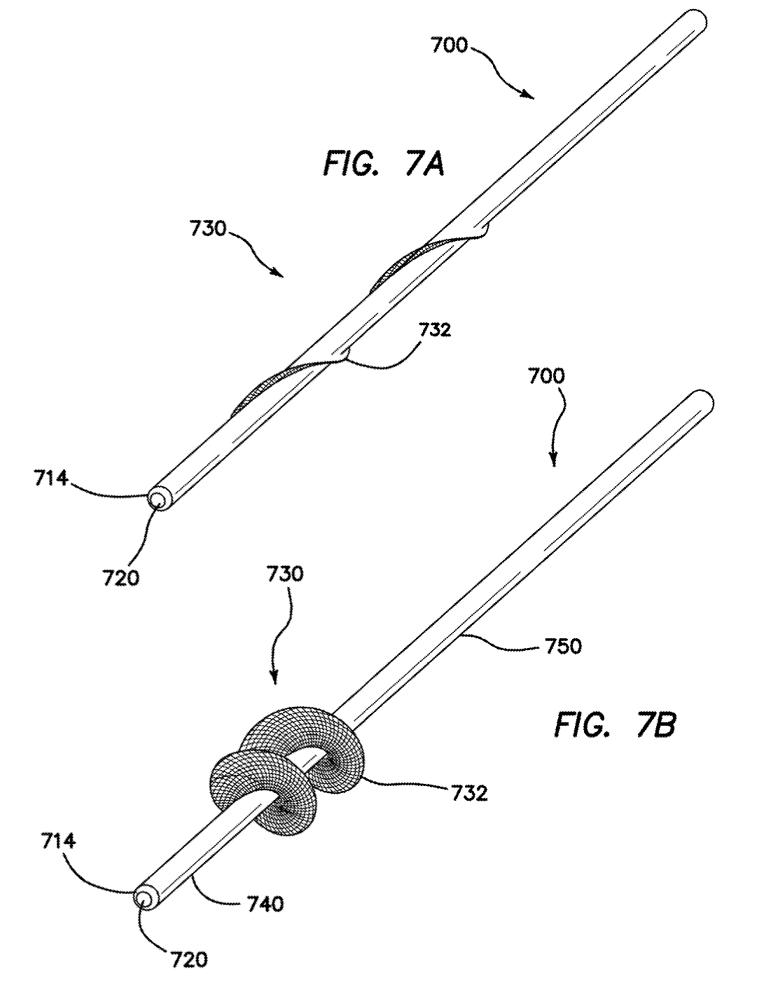

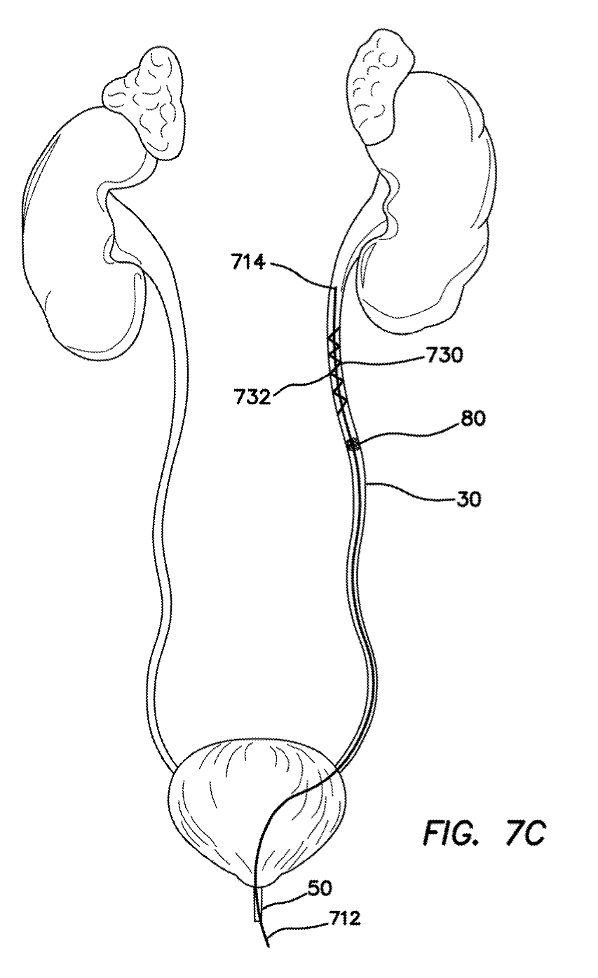

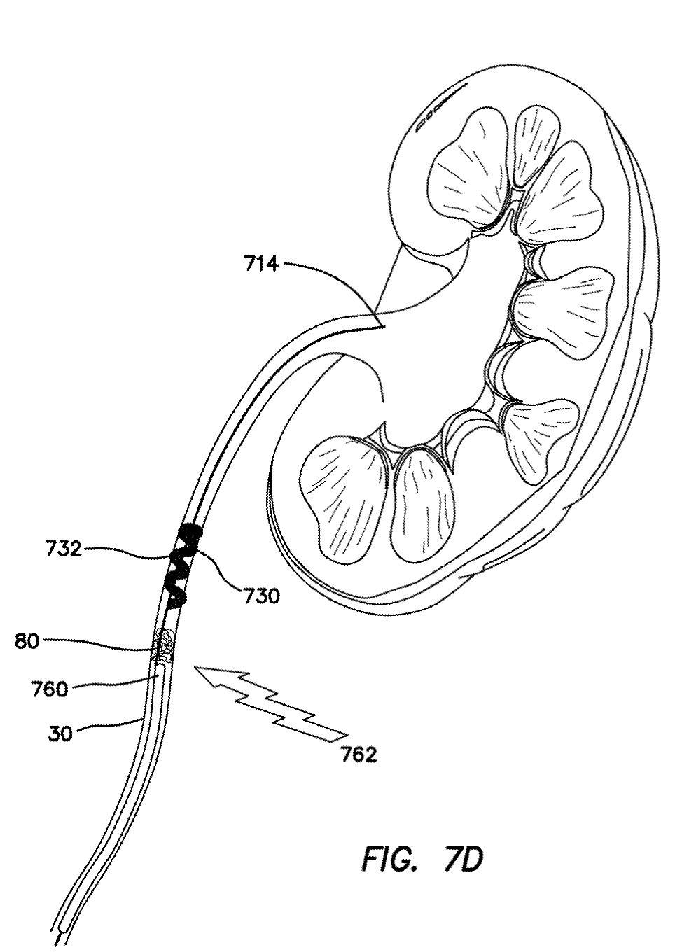

FIG. 7A illustrates in perspective view a distal end 714 of another embodiment of a catheter 700 with an expandable portion 730 in a low-profile state. FIG. 7B is a perspective view of the distal end 714 with the expandable portion 730 in a high-profile state. The catheter 700 is generally similar to the embodiments described above and illustrated in FIGS. 4A-6. In the illustrated embodiment, the expandable portion 730 comprises a generally helical or spiral deformable-film or deformable-mesh structure 732. Applying tension and/or torsion to the film or mesh structure 732, for example, by adjusting the relative positions of an inner tube 740 and an outer tube 750 coupled thereto as described above, maintains or converts the expandable portion 730 to the low-profile state illustrated in FIG. 7A. Releasing and/or reversing the tension or torsion converts the expandable portion 730 to the high-profile state illustrated in FIG. 7B, thereby wrinkling the film or mesh structure 732, which occludes or partially occludes the ureteral lumen 30, as illustrated in FIGS. 7C and 7D. Although the illustrated embodiment does not totally occlude the lumen 30, the torturous pathway created by the convoluted expandable portion 730 prevents upstream migration of ablated fragments of the ureteral mass 80.

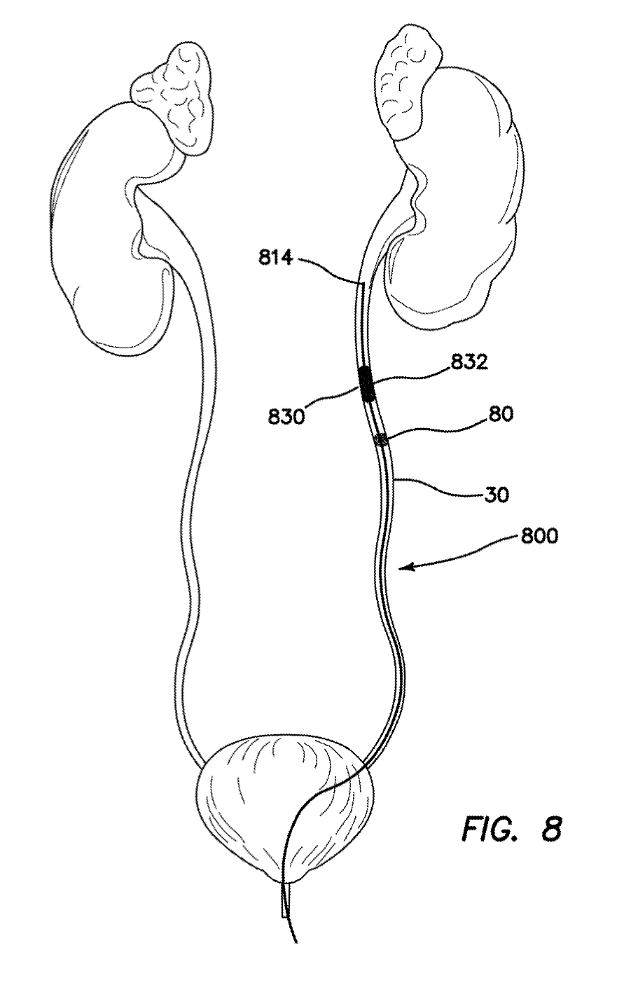

FIG. 8 is a view of a urinary tract with another embodiment of a catheter 800 placed therein with an expandable portion 830 in a high profile state deployed distally of a ureteral mass 80. The catheter 800 is generally similar to the embodiments described above and illustrated in FIGS. 4A-4C except that the expandable portion 830 comprises a compressible fabric 832. The expandable portion 830 is converted to the high-profile state by compressing and/or twisting the compressible fabric 832.

In another embodiment of a renal catheter (not illustrated), the expandable portion comprises a braided mesh element or a compressible fabric element, and at least one balloon disposed distally thereof.

Use of the embodiments of the catheters illustrated FIGS. 4A-8 in removing a ureteral mass is similar to the use of the embodiment illustrated in FIGS. 2A-2D. The following description references the embodiment illustrated in FIGS. 7C and 7D, but is applicable to all of the embodiments illustrated in FIGS. 4A-8. After placing the expandable portion 730 of the catheter distal of the ureteral mass 80 with the expandable portion 730 in the low profile state, the film or mesh structure 730 is deployed, thereby converting the expandable portion 730 to the high-profile state, as illustrated in FIGS. 7C and 7D. In some embodiments, placing the catheter 700 and/or deploying the expandable portion 730 is visually monitored. A lithotripter 760, for example, a laser lithotripter, is then activated, thereby emitting energy 762 that fragments the mass 80. In some embodiments, the mesh or film structure 732 is not appreciably damaged by the emitted energy 762. The deployed mesh or film structure 732 prevents fragments of the mass 80 from migrating upstream.

An irrigating fluid is introduced into the through lumen 720 at the proximal end 712 of the catheter and exits at the distal end 714 of the catheter, which is upstream of the ureteral mass 80 and the deployed expandable member 730 of the catheter. The bolus of irrigation fluid is placed within the collecting system, under very low pressure. The fluid “leaks” retrograde through the expandable member 730, which in the illustrated embodiment does not completely occlude the ureter 30. The fluid urges ablated fragments from the ureteral mass 80 proximally towards a proximal exit path 50. In some embodiments, the fluid and fragments are collected as described above. Adding fluid to the lumen distal of the mass 80 also helps in maintaining a safer ambient temperature, which is especially important when using a laser lithotripter.

Although this invention has been described in certain specific embodiments, many additional modifications and variations would be apparent to those skilled in the art. It is therefore to be understood that this invention may be practiced otherwise than specifically described, including various changes in the size, shape and materials, without departing from the scope and spirit of the present invention. Thus, embodiments of the present invention should be considered in all respects as illustrative and not restrictive, the scope of the present invention to be determined by the appended claims and their equivalents rather than the foregoing description.

1. A catheter based occlusive device for use within a body lumen to maintain a gradient fluid pressure and form a luminal blockage, comprising:
an elongate luminal catheter body having a lumen extending from a proximate end to a distal end of the catheter body; and
a plurality of expandable portions near the distal end of the catheter body;
wherein in the low-profile state, each of the expandable portions has a crossing profile sufficient to permit placement of the expandable portion distal of a ureteral mass, and in the high-profile state, each of the expandable portions substantially block fragments generated by lithotripsy of the ureteral mass from traveling distally thereof.

2. The catheter of claim 1 wherein the plurality of expandable portions are constructed of expandable cylindrically woven mesh fabric mounted around the catheter body.

3. The catheter of claim 2 wherein the expandable cylindrically woven mesh fabric is formed as a cone.

4. The catheter of claim 2 wherein the expandable cylindrically woven mesh fabric is formed as a ball.

5. The catheter of claim 2 wherein the expandable cylindrically woven mesh fabric is formed as an ellipsoid.

6. The catheter of claim 2 wherein the expandable cylindrically woven mesh fabric is formed as a cylinder disposed between a proximally facing cone and a distally facing cone.

7. The catheter of claim 6 wherein the cylinder has a diameter substantially smaller than the proximally facing and distally facing cones.

8. The catheter of claim 1 wherein the plurality of expandable portions are constructed of inflatable balloons concentrically mounted around the catheter body.

9. The catheter of claim 1 further comprising:
an deployment actuator coupled to the plurality of the expandable portions and disposed at the proximal end of the catheter body; and
wherein the deployment actuator reversibly converts the plurality of the expandable portions from a low profile state and a high profile state.

10. The catheter of claim 1 wherein the plurality of the expandable portions comprises an expandable braided mesh with a helical shape winding around a longitudinal axis of the catheter body.

11. The catheter of claim 9 wherein the expandable braided mesh comprises at least one of a polymer, polyester, polyamide, polytetrafluoroethylene, and polyurethane.

12. The catheter of claim 1 wherein the plurality of the expandable portions comprises a compressible fabric.

13. The catheter of claim 9 wherein the deployment actuator comprises rotatable coaxial tubes rotationally interacting to reversibly convert the plurality of expandable portions from a low-profile to a high-profile state.

14. The catheter of claim 1 further comprising a lithotripter directing focused energy upon an obstruction to form fragments

15. The catheter of claim 1 further comprising a removable ureteral stent surrounding the catheter body and the plurality of expandable portions.

16. The catheter of claim 1 further comprising a fluid aspirator coupled to the lumen of the catheter body to partially fill a distal portion of the body conduit with fluid.

17. A catheter based occlusive device for use within a body lumen to maintain a gradient fluid pressure and form a luminal blockage, comprising:
an elongate luminal catheter body having a lumen extending from a proximate end to a distal end of the catheter body; and
a plurality of expandable portions attached to the distal end of the catheter body, the plurality of expandable portions having a low-profile state and a high-profile state;
wherein the lumen of the catheter body is a fluid conduit to inject irrigation fluid out the distal end of the catheter body and distally from the plurality of expandable portions and the plurality of expandable portions in the high-profile state obstructs fragments of a ureteral mass from traveling distally thereof and does not hinder the fluid conduit.

18. The catheter of claim 17 wherein the catheter body includes a second lumen extending from the proximate end to a distal end of the catheter body and being an actuation passage through which an actuation connector extends to move at least one of the plurality of expandable portions from a low-profile state to a high-profile state and a third lumen extending from the proximate end to the distal end of the catheter body and being a visualization passage through which an optical imaging connector extends to illuminate and visualize the ureteral mass.

19. The catheter of claim 17 wherein at least one of the plurality of expandable portions is one-way fluid permeable in a proximal direction.

20. The catheter of claim 17 wherein a proximal expandable portion of the plurality of expandable portions is destructible by a laser lithotripter and a distal expandable portion is not destructible by a laser lithotripter.
